(19) United States
(12) Patent Application Publication
(10) Pub. No.: US 2003/0166006 A1
(43) Pub. Date: Sep. 4, 2003

(54) Glucose-1-phosphate thymidylyltransferase and method for selecting inhibitors thereof

(76) Inventors: James Naismith, St. Andrews (GB); Wulf Blankenfeldt, St. Andrews (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/332,935

(22) PCT Filed: Jul. 13, 2001
(86) PCT No.: PCT/GB01/03152

(30) Foreign Application Priority Data
Jul. 15, 2000 (GB) 0017323.7

(51) Int. Cl. G01N 33/53; G06F 19/00; G01N 33/48; G01N 33/50

(52) U.S. Cl. 435/7.1; 702/19

(57) ABSTRACT

There is provided a method of obtaining selecting agents which inhibit the enzyme glucose-1-phosphate thymidylyltransferase (Rm1A) based upon analysis of a model of the active and regulatory site (s) of Rm1A and interaction therewith by a potential inhibitory agent. The invention is based upon the provision of information on the structure of Rm1A obtained through X-ray diffraction studies since a crystallised form of Rm1A was obtained for the first time. The purified and crystallised from of Rm1A, obtained from Psemdomononas aeruginosa is also described.

The present invention relates to the enzyme glucose-1-phosphate thymidylyltransferase (RmlA) and its use in a method of selecting for agents which inhibit the enzyme glucose-1-phosphate thymidylyltransferase (RmlA).

Bacterial cell-surface glycoconjugates are essential for survival of pathogenic bacteria and for interactions between bacteria and the host. Consequently, there is reason to believe that inhibitors directed against target reactions in assembly of the cell-surfaces glycoconjugates may provide viable alternate therapeutic approaches. However, bacterial cell-surface glycoconjugates show remarkable structural diversity due to variations of the sugar components, linkages and substitutions. A successful strategy requires identification of enzymes and pathways unique to bacteria, yet present within a wide spectrum of bacterial species. One such target is the synthesis of the activated form of L-rhamnose, dTDP-L-rhamnose.

L-rhamnose is a 6-deoxyhexose that is found in the cell wall of many pathogenic microorganisms. In Gram-negative bacteria it is one of the important residues of the O-antigen of lipopolysaccharide, a factor which is a key determinant for the virulence of these species. In Pseudomonas aeruginosa, this sugar is a constituent of the core oligosaccharide and serves as the receptor for O-antigen polymer (Rahim et al., 2000). Gram-positive bacteria such as streptococci and mycobacteria, on the other hand, utilise L-rhamnose in the arabinogalactan (AG) that attaches the lipid mycolic acid layer to the peptidoglycan layer (McNeil et al., 1990). It has been demonstrated that this attachment is of vital importance to mycobacteria: inhibitors of the formation of the arabinan portion of AG, e.g. ethambutol, can stop cell growth and are effective drugs (see for example Deng et al., 1995). As L-rhamnose is not found in mammals inhibition of its biosynthetic pathway is a target of interest in the development of novel antibiotics.

Four enzymes, glucose-1-phosphate thymidylyltransferase (RmlA), dTDP-D-glucose 4,6-dehydratase (RmlB), dTDP-6-deoxy-D-xylo-4-hexulose (RmlC) and dTDP-6-deoxy-L-lyxo 4-hexulose-4-reductase (RmlD) are required for the synthesis of dTDP-L-rhamnose from α-D-glucose-1-phosphate (G1P) and dTTP. Significantly, these proteins are highly conserved amongst microorganisms (see for example Ma et al., 1997; Graninger et al., 1999) and therefore conclusions drawn from the structure of a protein from one species will have strong implications for the corresponding enzyme of another origin.

Pseudomonas aeruginosa is a Gram-negative bacterium that colonises many children with cystic fibrosis where it is a significant cause of morbidity and mortality. In addition it is an opportunistic pathogen that can cause a wide variety of infections, particularly in victims of severe burns and in patients who are for any reason immunosuppressed. This makes Pseudomonas aeruginosa one of the most prevalent pathogens in hospital-acquired infections. Due to its high resistance to antibiotics it is a particularly dangerous pathogen and any approach towards its control is highly sought.

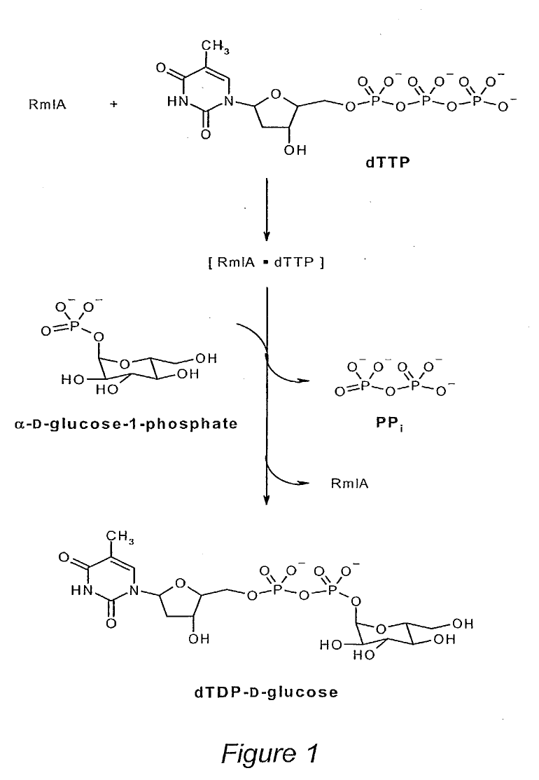

RmlA (glucose-1-phosphate thymidylyltransferase, E.C. 2.2.7.24) catalyses the first of four steps in the transformation of glucose-1-phosphate (G1P) to 2′-deoxy-thymidylyl-diphospho-L-rhamnose (dTDP-L-rhamnose or dTDP-Rha) in a Mg²⁺ dependent manner (see FIG. 1). The reaction product, 2′-deoxy-thymidylyl-diphospho-D-glucose (dTDP-Glc), can also be back-pyrophosphorylysed by RmlA, yielding 2′-deoxy-thymidylyl-triphosphate (dTTP) and GlP. It has been demonstrated that the enzymatic activity of RmlA is allosterically regulated by dTDP-Rha (Melo & Glaser, 1965), making the protein a key player in the biosynthesis of rhamnose. Hence, it presents itself as an attractive target in the development of novel antibiotics.

RmlA is of particular interest as it is not only involved in the biosynthesis of L-rhamnose but also in the pathways leading to other 6-deoxy sugars such as L-talose or D-fucose as these share common intermediates in the conversion of D-glucose to their end-products (Nakano et al., 2000; Yoshida et al., 1999). The enzyme is highly homologous to other bacterial sugar nucleotide transferases (e.g. glucose-1-phosphate uridylyltransferase). The sugar nucleotide transferases catalyse the first step in all sugar nucleotide chemistry and are of key importance in biology and biotechnology. As RmlA shows no sequence relationship to any protein structure currently deposited in the PDB (Sussman et al., 1998), it is expected to contain a novel fold. It is not yet fully clear which reaction mechanism RmlA and related enzymes follow. They may either obey Theorell-Chance (Theorell & Chance, 1951) or ordered sequential bi-bi kinetics with the nucleotide triphosphate binding to the protein first (Paule & Preiss, 1971). An oxygen of GlP's phosphate group is then believed to carry out a nucleophilic attack on the NTP α-phosphate group directly as has been demonstrated by the inversion of stereochemistry on thiosubstituted NTPs (Sheu & Frey, 1978; FIG. 1).

Purification and crystallisation of RmlA enzymes from various micro-organisms has been generally possible but the crystals obtained proved to be of insufficient quality for structural studies, such as X-ray diffraction studies. Surprisingly, it has now been found that it is possible to obtain crystals of RmlA from Pseudomonas aeruginosa, and that crystals are of sufficient quality to perform structural studies.

The present invention provides a purified and crystallised form of RmlA from Pseudomonas aeruginosa and its X-ray structure.

The present invention further provides a method of selecting agents which inhibit the enzyme glucose-1-phosphate thymidylyltransferase (RmlA), said method comprising the steps of:

a) providing a model of the active or regulatory site(s) of RmlA;

b) reviewing the structure of a potential inhibitory agent for at least one of these sites; and

c) analysing the potential interaction of said agent in said site(s).

Optionally, said model may be in the form of a computer data or graphic file, and will usually be based upon the X-ray crystal co-ordinates of RmlA. Numerous computer programs, including graphic programs, now exist to facilitate the handling of said X-ray crystal co-ordinates and mention may be made of FRODO (version O), Insight and SYBIL, and the like.

Conveniently, the potential inhibitory agent may itself be reviewed in the form of a model, for example a computer data file. Thus, the interaction between the enzyme and potential inhibitory agent can be analysed through interaction of the models, and conveniently may be calculated by computer.

The structure of the agent to be tested for RmlA inhibitory activity may conveniently likewise be reviewed and analysed in the form of X-ray crystal co-ordinates or approximations thereof. Optionally, the potential intermolecular interaction between the agent under test and the active site of RmlA will be analysed with the aid of a computer.

In a further embodiment, the present invention provides a method of selecting an anti-microbial (such as anti-bacterial or anti-fungal) compound, said method comprising following the steps a) to c) outlined above, and including the step of selecting an agent that binds to an active or regulatory site of RmlA sufficiently tightly to impede the biosynthesis of rhamnose and thus growth of the micro-organism. It is preferred that the anti-microbial agent is particularly effective against Pseudomonas aeruginosa.

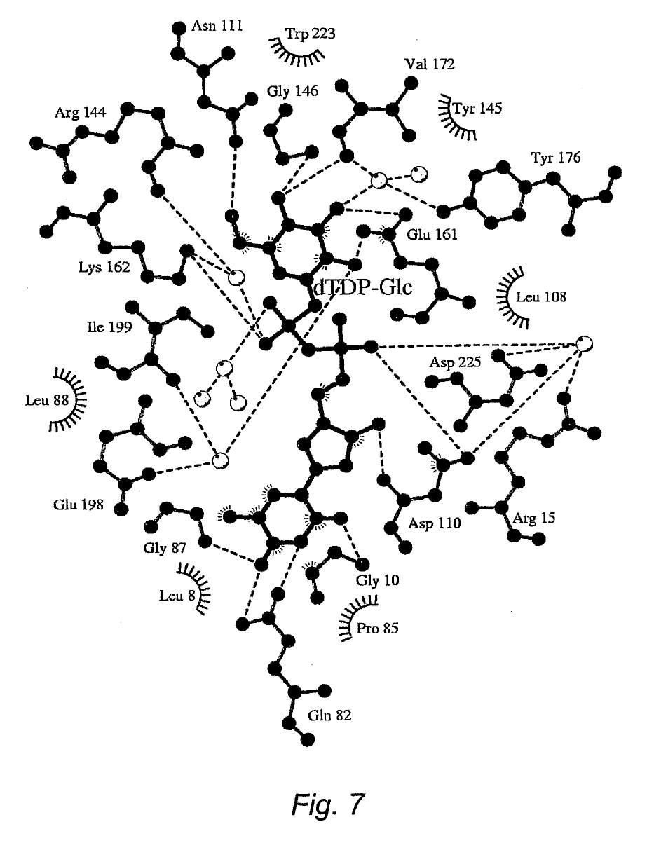

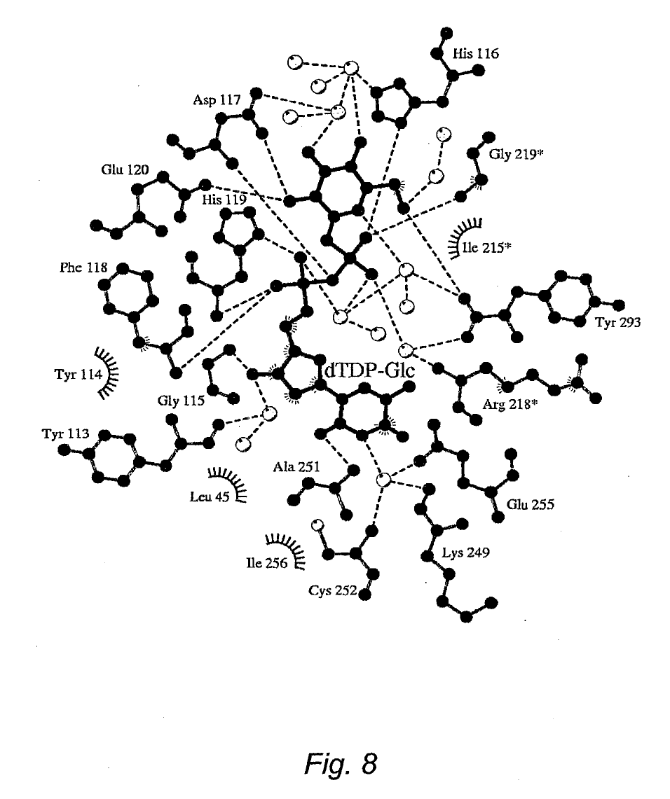

In a preferred embodiment, the agent will include one or more regions able to interact with one or more of the amino acids of the active or regulatory sites (and in particular the amino acids specifically mentioned in the description of the active and regulatory sites given below and in FIGS. 7 and 8) to impede the biosynthesis of rhamnose.

For example, the agent may desirably comprise a negative charge and the interaction with the active site of RmlA will desirably include an association between the negative charge of the agent and at least one of the amino acids Arg 15 and Lys 25.

The agent may also be provided with thymidyl-like moiety able to interact (e.g. form hydrogen bonds) with Gly 10, Gln 82 and/or Gly 87.

The agent may also be provided with a glucose-like moiety able to interact (e.g. form hydrogen bonds) with Asn 111, Gly 146, Glu 161, Val 172 and/or Tyr 176.

The present invention will now be further described with reference to the examples and figures in which:

Figure Legends

FIG. 1: Shows the first step of the conversion of glucose-1-phosphate (G1P) into 2′-deoxy-thymidylyl-diphospho-L-rhamnose or DTP-L-rhamnose. The reaction catalysed by RmlA transforms G1P and dTTP to dTDP-D-glucose.

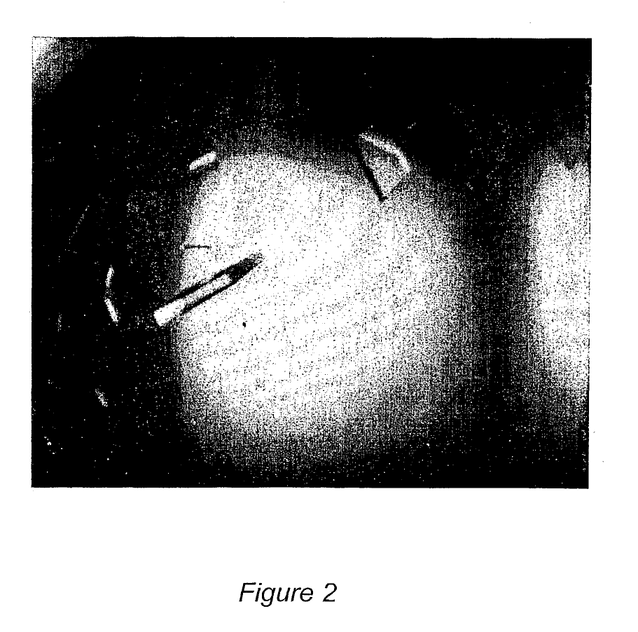

FIG. 2: Is a photograph of RmlA crystals obtained. These crystals have been grown in the presence of dTMP.

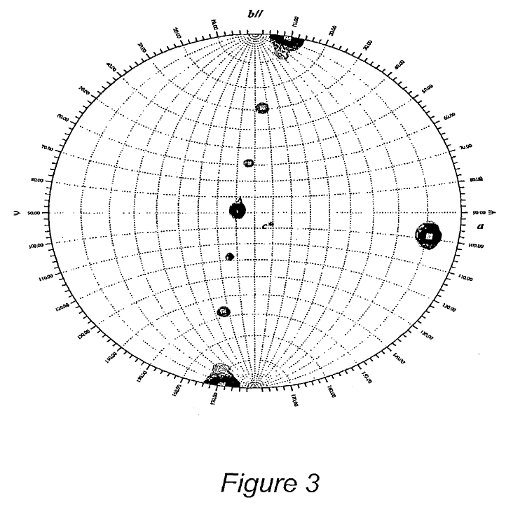

FIG. 3: κ=180° section of the self-rotation search in the TMP dataset. Done with REPLACE (Tong & Rossmann, 1997). Search angle: polar XYK; orthogonalisation AXABZ

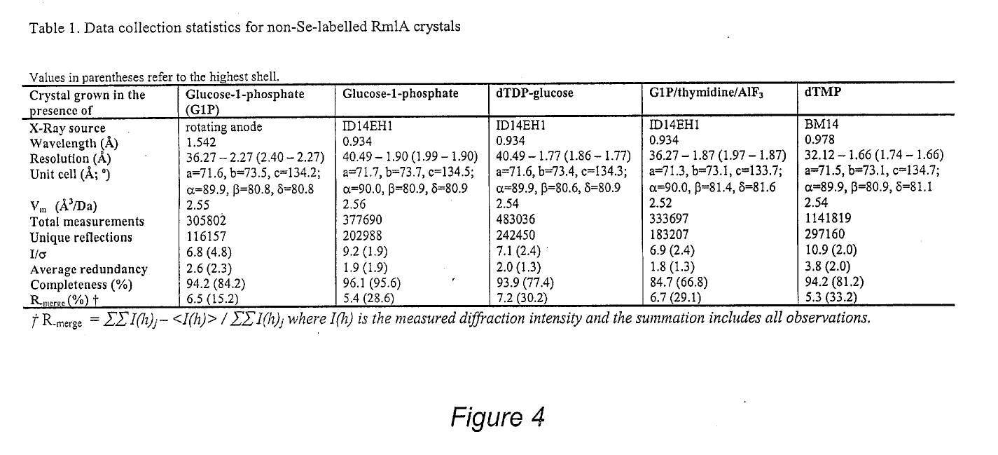

FIG. 4: Table 1: Data collection statistics for non-Se-labelled RmlA crystals.

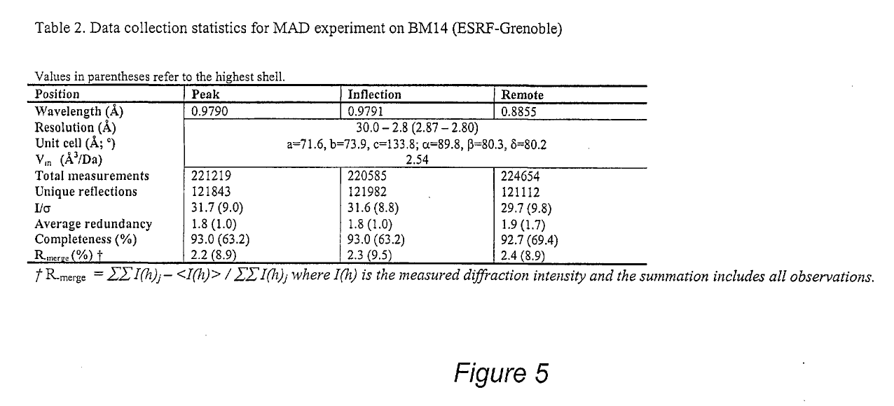

FIG. 5: Table 2: Data collection statistics for MAD experiment on BM14.

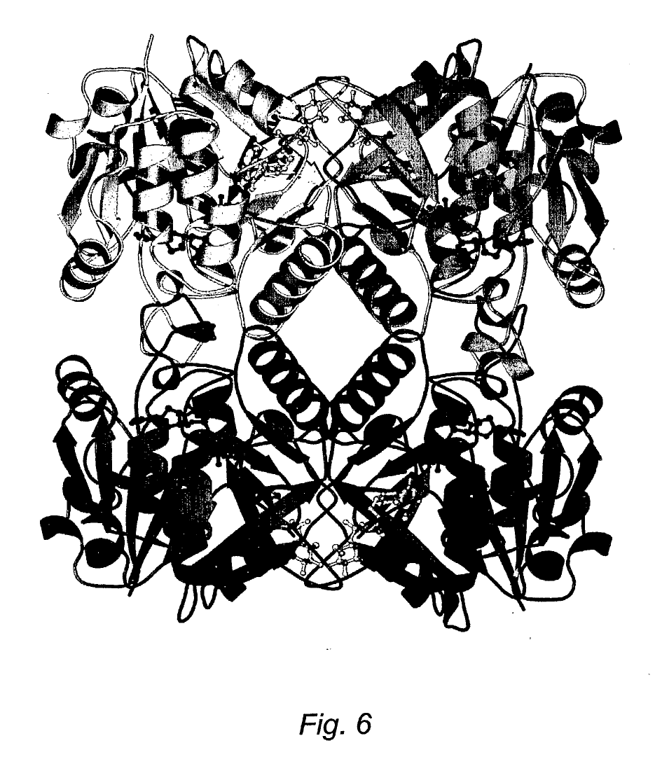

FIG. 6: Overall structure of the RmlA tetramer with the location of active (dark) and regulatory (light) binding site indicated by bound ligand. The bound molecule is dTDP-Glc in all cases.

FIG. 7: Interactions of dTDP-Glc in the active center of RmlA. The hydrogen bonding network is indicated by dashed lines. Hydrophobic contacts are shown as lunes. Water residues are presented as cyan spheres.

FIG. 8: Interactions of dTDP-Glc in the regulatory binding site of RmlA.

### EXAMPLES

RmlA Overexpression and Purification

The open reading frame of the gene encoding RmlA from Pseudomonas aeruginosa was amplified using PCR with primers that incorporated a 5′ NcoI and a 3′ BamHI site to facilitate cloning into a modified pET23a(+) vector (Newton & Mangroo, 1999). The plasmid also contained a sequence coding for a 6×His-tag on the N-terminus of RmlA to allow an easy purification on metal chelating columns. Expression involves the IPTG (isopropyl-β-D-thiogalactoside)-inducible T7 promotor and ribosome-binding sites conferred by the vector. The sequence of the amplified and cloned gene was confirmed to be identical to the chromosomal copy excepting the N-terminal 6×His-tag.

In order to overexpress RmlA E. coli BL21(λDE3) cells transformed with the plasmid were grown at 310 K in Luria-Bertani medium containing 100 μg ml⁻¹ ampicillin until the OD₆₀₀ reached 0.6-0.8. Expression of the protein was then induced by addition of 1 mM IPTG. After further 3 h of culture cells were harvested by centrifugation (20 min, 6,000 g, 277 K).

The cell pellet was resuspended in a lysis buffer containing 20 mM Tris-HCl pH 8.5, 100 mM NaCl, 0.2 mM DTT, 5 mM PMSF and 100 μg ml⁻¹ hen egg white lysozyme. After 30 min incubation at room temperature, the viscosity of the mixture was decreased by the addition of DNAse I (20 μg ml⁻¹) and by sonication (five cycles of 1 min interrupted by 1 min periods on ice). The suspension was centrifuged at 20,000 g and 277 K for 20 min and the supernatant then brought to 20% ammonium sulfate saturation. After incubation on ice for 1 h a second centrifugation (20 min, 20,000 g, 277 K) was carried out and the supernatant then dialysed against two changes of 1 litre 20 mM Tris-HCl pH 7.0, 20 mM imidazole and 500 mM NaCl. The filtered protein solution was passed through a POROS-MC column which had been pre-loaded with nickel sulphate. Proteins were eluted with a 20 to 500 mM imidazole gradient. A protein with a molecular weight corresponding to RmlA (˜34 kDa) was found in a peak eluting at approx. 200 mM imidazole. Fractions corresponding to this peak were pooled, concentrated with a 10 kDa cut-off Amicon membrane and dialysed against two changes of 1 litre of 20 mM Tris-HCl pH 8.5 at 277 K containing 10 mM EDTA in the first change to remove contaminating nickel ions. For further purification the protein was applied to a POROS-HQ anion exchange column on a BioCAD 700E Workstation. Elution was achieved with a 50 to 1000 mM NaCl gradient. RmlA eluted at a salt concentration of 200 mM. Pooled fractions were brought to a protein concentration of approx. 4 mg ml⁻¹ as determined by Bradford assay (Bradford, 1977) using bovine serum albumin as standard and then dialysed against two changes of 50 mM Tris-HCl pH 7.5 at 277 K. Prior to crystallisation experiments DTT was added to 4 mM and the solution filtered through a 0.2 μm membrane. This procedure typically yielded 30 mg of pure protein per litre of bacterial culture. Small aliquots of the purified protein could be stored at 255 K without deterioration for several months without addition of cryoprotecting agents. Selenomethionine labelling of Pseudomonas aeruginosa RmlA could not be achieved in met⁻ B834(λDE3) E. coli cells. Under all conditions tested the protein formed inclusion bodies. Selenomethionine enriched protein was therefore produced by inhibition of the methionine biosynthesis pathway in E. coli BL21(λDE3) (Doublie, 1997). Briefly, cells were grown in M9 medium (64 g l⁻¹ Na₂HPO₄.7H₂O, 15 g l⁻¹ KH₂PO₄, 2.5 g l⁻¹ NaCl; 5 g l⁻¹ NH₄Cl, 1 mM MgSO₄, 0.4% glucose, 0.1 mM CaCl₂) at 310 K until OD₆₀₀ reached 0.6. At this stage the amino acids lysine, phenylalanine and threonine were added to final concentrations of 100 mg l⁻¹ and isoleucine, leucine and valine to 50 mg l⁻¹. Seleno-L-methionine was added to a concentration of 60 mg l⁻¹. The temperature was lowered to 303 K and the culture left to shake for further 15 min before protein overexpression was induced with 1 mM IPTG. After 6 h cells were harvested and lysed as described above. 13 mg pure protein per litre of culture could be isolated.

Protein Analysis

Following the two HPLC steps, the protein appeared to be pure as judged by a SDS silver nitrate stained gel (single band at an apparent molecular weight of 34 kDa); the calculated molecular weight based on sequence being 33773 Da. A single peak with a molecular weight of 33803 Da was found in the MALDI mass spectrum. Dynamic light-scattering results (DynaPro 801) indicated the native protein to be monodisperse with a molecular weight in the range of 106 to 122 kDa indicative of a trimeric or tetrameric protein. N-terminal sequencing was performed and confirmed the protein to be Rm1A.

The efficiency of the selenomethionine labelling procedure was scrutinised by MALDI mass spectrometry. A shift of +304 Da was found for the intact labelled protein corresponding well to the predicted additional mass of 282 Da (6 methionine residues per chain). In an important and useful second check, sulfur methionine containing fragments were undetectable in the MALDI mass spectrum of a tryptic protein digest.

RmlA Crystallisation

Initial crystallisation conditions were obtained from Screen I and II of Hampton Research (Jancarik & Kim, 1991; Cudney et al., 1994) plus NaCl, PEG 6000, PEG 6000/lithium sulphate and MPD grids. The sitting drop vapour diffusion method (Ducruix & Giegé, 1992) with 4 μl of protein sample and 4 μl of precipitant at 293 K was used throughout. Crystals appeared under 27 of the initial 192 conditions, in some cases 10 min after setup. Most promising were results from the PEG 6000/lithium sulphate grid and hence these conditions were further optimised. Plate type crystals of approx. 0.3×0.3×0.05 mm size (FIG. 2) were obtained after one to seven days using 9 to 12% (w/v) PEG 6000, 0.5 M lithium sulphate and 0.1 M citrate/NaOH pH 4.6 as precipitating solution. The initial very high mosaicity of these crystals could be greatly reduced by the addition of 1 to 2 μl 10 to 50 mM G1P, dTMP or dTDP-glucose to the protein prior to crystallisation.

Data Collection

A 2.2 Å resolution dataset (see Table 1, FIG. 4) from a single flash frozen crystal grown in the presence of G1P was collected in-house at 110 K using a Nonius/MacScience DIP2000 imaging plate detector system. Data were recorded as 245 non-overlapping 20 min 1° oscillations. Cryoprotection was achieved by washing the crystal in mother liquor supplemented with 16% (v/v) PEG 600 for 10 to 15 s. The oscillation images were indexed and integrated with the program MOSFLM (Leslie, 1992) and scaled with the CCP4 program SCALA (CCP4, 1994). Higher resolution datasets of crystals grown in the presence of G1P, dTMP, dTDP-glucose or thymidine/glucose-1-phosphate/AlF₃ were measured at the ESRF-Grenoble at beamlines ID14EH1 and BM14 (Table 1). All crystals were triclinic with approximate cell parameters of a=71 Å, b=73 Å, c=134 Å; α=89.9°, β=81° and γ=81°. All attempts to index or reduce the data in a higher spacegroup failed, a native Patterson map shows no non-origin peak. A majority of the crystals were actually twinned. This could only be detected from the diffraction pattern. Trial and error was used to locate single crystals for analysis. Flash-annealing with the crystal remaining in the loop (Yeh & Hol, 1998) in some cases helped to achieve a less mosaic diffraction pattern.

In addition to the datasets shown in Table 1 (FIG. 4) a three-wavelength MAD experiment with a selenomethionine labelled crystal that was grown in the presence of G1P was carried out at beamline BM14 of the ESRF-Grenoble The crystal-to-detector distance was adjusted so that the outer rim of the detector area corresponded to a resolution of 2.8 Å. 730 non-overlapping 0.5° oscillations were recorded at each of three wavelengths. The three wavelengths were chosen from an EXAFS scan of the crystal to correspond to the maximum of f″ (peak), the maximum modulus of f′ (inflection) and minimum modulus of f′ (remote). These data were indexed and integrated with DENZO and scaled with SCALEPACK (Otwinowski & Minor, 1996) and are shown in Table 2 (FIG. 5).

Preliminary Structural Characterisation

At the beginning of this study it was not clear whether native RmlA is a trimeric or a tetrameric protein. A self-rotation search of the TMP dataset with REPLACE (Tong & Rossmann, 1997) reveals three major (>30 σ) plus several minor (approx. 10 to 15 σ) twofold axes (FIG. 3). In addition, a 60°- and a 120°-rotation axis (15.4 and 14.6 σ) are found lying parallel to the major 180°-rotation axes at φ=8° and ψ=98° (data not shown). The interpretation of these results was greatly aided by the determination of selenium atom positions with the program SOLVE 1.17 (Terwilliger & Berentzen, 1999). Twenty-four sites were found which could easily be grouped into eight equivalent clusters of three atoms. The clusters fall into two sets of four indicating that RmlA is a tetramer and that the unit cell of the P1 crystal form contains two tetrameric molecules. The rotation superimposing the two tetramers can be described as either as a 60°-, a 120°- or a 180°-rotation axis depending on which monomer is used as a reference point. This explains the existence of major and minor twofold axes in the κ=180° self-rotation search. First, there are inter- and intra-tetramer 180°-axes lying parallel to each other. Second, in the case of exclusively intra-tetramer axes only two pairs of two monomers are superimposed while in the other the intramolecular vectors of eight protein chains contribute to the peak in the self rotation function, leading to a stronger signal.

The asymmetric unit of the crystal contains approximately 2400 amino acid residues and has a solvent content of 51%, corresponding to a V_{M} of 2.54 Å³ Da⁻¹ (Matthews, 1968).

A partial set of co-ordinates from Pseudomonas aeruginosa RmlA is listed in Annex 1. The co-ordinates are given in two sections; the first section gives all atoms up to a distance of 15 A to the bound product in the active site; and the second section gives all atoms up to a distance of 15 Å to the bound product in the regulatory site. The data is derived from the dTDP-glucose dataset given in FIG. 4 (table 1) and represent a model of excellent geometrical properties with an R-factor of 16.3% and an R_{free} of 21.8% at a resolution of 1.77 Å. The co-ordinates also contain one bound molecule of dTDP-glucose in each monomer's active centre, which can be used in computer programs for inhibitor modelling.

Structural Characteristics

Fold

RmlA is a 222 tetrameric molecule and its structure is represented in FIG. 6. In Pseudomonas aeruginosa the monomer has a chain length of 293 amino acids. The subunit's fold can be described as a single domain αβα sandwich, meaning that a central β-sheet is covered by layers of helices from both sides. In RmlA this mixed β-sheet is seven stranded in the order 3214657 with strand 6 being antiparallel to the rest. In addition, both helical layers contain a two stranded β-sheet structure as well. Due to its tetrameric nature each monomer is in contact with two neighbouring subunits.

Binding Sites

The Rm1A monomer is capable of binding two molecules of dTDP-Glc. By sequence comparison with related nucleotidyltransferases and inspection of the glucose 1-phosphate co-complex, it is possible to definitively assign the active centre to the areas around the black bound molecules in FIG. 6. The second binding site (light grey molecules in FIG. 6) is likely going to be involved in allosteric regulation of RmlA's enzymic activity.

Active Site

The active site is exclusively made up of amino acids from one monomer. FIG. 7 gives a schematic representation of the most important interactions between dTDP-Glc and the enzyme. The amino acids can be subdivided into three groups.

Group one contains the residues involved in the catalytic mechanism and in particular the formation or pyrophosphorolysis of diphospho ester bonds. Their importance is highlighted by a high degree of conservation amongst nucleotidyltransferases. These residues are Arg15, Asp110, Lys162 and Asp225. A high degree of conservation is also observed for Lys25 (not shown in FIG. 7). The positively charged Arg15 and Lys25 are responsible of binding the β- and the γ-phosphate group of dTDP as can be concluded from an additional sulphate molecule that is bound in the active site of RmlA but not shown in FIG. 7. The position of Lys25 is stabilised by a salt bridge with Asp110, another highly conserved residue in sugar nucleotidyltransferases. The importance of Lys162 in the active centre lies in binding of the phosphate group of glucose-1-phosphate. It ensures correct orientation towards dTTP for nucleophilic attack on the α-phosphate group.

Groups 2 and 3 provide specificity for thymidyl and/or glucosyl ligands, respectively. Specificity for the thymidyl moiety results from Gly10, Gln82 and Gly87 which all form hydrogen bonds with the pyrimidine ring The glucose part of RmlA substrates is hydrogen bonded to Asn111, Gly146, Glu161, Val172 and Tyr176. Among these, the chelating interaction of Glu161's side chain will be of high importance as it can only bind to hydroxyl groups of the sugar if these are in equatorial position.

A hydrophobic patch of three leucine residues (Leu8, Leu88, Leu108) lines the active site from the bottom. Other residues in only hydrophobic interaction are Pro85, Tyr145 and Trp223.

Regulatory Site

The second binding site for dTDP-Glc is located in the interface between two monomers (FIG. 6), hence amino acids from two subunits contribute to its formation. The residues in this binding site (FIG. 8) are not conserved in more distantly related nucleotidyltransferases. Therefore, these enzymes' allosteric control might be achieved by other mechanisms. However, glucose-1-phosphate thymidylyltransferases from other organisms will have binding sites similar to FIG. 8 as can be concluded from their amino acid sequences. It can be concluded from FIG. 8 that dTDP-Glc is not the natural ligand of this binding site as most contacts between the protein and the glucosyl moiety are mediated by water molecules whilst the remainder of the ligand shows mainly direct hydrogen bonding. Suitable inhibitors may either bind to the active site of RmlA, acting in a competitive mode to natural substrates and being non-cleavable, or may exploit the allosteric properties of RmlA. In the case of RmlA from Pseudomonas aeruginosa the latter might be the preferred approach: the protein is strongly inhibited by dTDP-rhamnose, the final product of the four enzyme pathway (Melo & Glaser, 1965), possibly by binding to the second binding site indicated in FIG. 6. As rhamnose is not found in mammals, dTDP-rhamnose derived compounds might provide lesser side effects in the application as antibiotics and are potentially good candidates as suitable RmlA inhibitors.

Several methods for essaying the activity of RmlA and related enzymes in both sugar mucleotide synthesis and pyrophosphorolysis directions have been described in the literature. They are normally based on the incorporation of radioactive compounds into the reaction products and seem to be less suited for the development of new inhibitors by high throughput screening as they require a time-consuming separation of the reaction mixtures. Therefore, it is proposed to use coupled enzyme assays for drug development purposes.

In the synthesis direction, the reaction can be followed by monitoring the production of pyrophosphate using pyrophosphate dependent fructose-6-phosphate kinase (PPᵢ-PFK). This enzyme generates fructose-1,6-diphosphate (F-1,6-DP) from fructose-6-phosphate (F-6-P) and pyrophosphate (PPᵢ). F-1,6-DP is then cleaved by aldolase to yield glyceraldehyde-3-phosphate (GAP) and dihydroxyacetone phosphate (DHAP). GAP is isomerised by triosephosphate isomerase (TPI) to give a second molecule of DHAP. Finally, DHAP is reduced to glycerol-3-phosphate (G3P) by glycerophosphate dehydrogenase (GDH) in an NADH-dependent reaction such that the production of pyrophosphate is coupled to the depletion of NADH which can be recorded by the decrease in absorption at 340 nm (O'Brien, 1976):

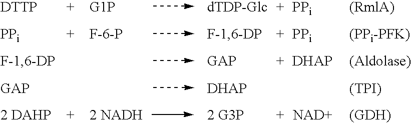

The pyrophosphorolysis direction can be monitored by following the production of G1P, which is then isomerised to glucose-6-phosphate by phosphoglucomutase (PGM) and subsequently oxidised to 6-phospho-gluconolactone (6PGL) by glucose-6-phosphate dehydrogenase (G6P-DH), thereby generating one molecule of NADPH. This can again be followed at 340 nm (Kornfeld & Glaser, 1961).

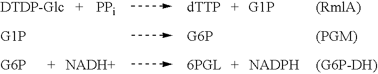

Both assays are rapid and easy to carry out.

The active and regulatory sites of Rm1A and interactions with their natural substrates are further illustrated by the Figures.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

### REFERENCES

Allard, S. T., Giraud, M. F., Whitfield, C., Messner, P. & Naismith, J. H. (2000). Acta Cryst. D56, 222-225

Bradford, M. M. (1976). Anal. Biochem. 72, 248-252.

Cudney, R., Patel, S., Weisgraber, K., Newhouse, Y., & McPherson, A. (1994). Acta Cryst. D50, 414-423.

Collaborative Computational Project, Number 4 (1994). Acta Cryst. D50, 760-763.

Deng, L., Mikusova, K., Robuck, K. G., Scherman, M., Brennan, P. J. & McNeil, M. R. (1995). Antimicrob. Agents Chemother. 39, 694-701

Doublie, S. (1997). Methods Enzymol. 276, 523-530

Ducruix, A. & Giegé, R. (1992). Crystallisation of Nucleic acids and Proteins, a Practical Approach. Oxford: IRL Press.

Giraud, M. F., Gordon, F. M., Whitfield, C., Messner, P., McMahon, S. A. & Naismith, J. H. (1999a). Acta Cryst. D55, 706-708

Giraud, M. F., McMiken, H. J., Leonard, G. A., Messner, P., Whitfield, C. & Naismith, J. H. (1999b). Acta Cryst. D55, 2043-2046

Graninger, M., Nidetzky, B., Heinrichs, D. E., Whitfield, C. & Messner, P. J. (1999). J. Biol. Chem. 274, 25069-25077.

Jancarik, J. & Kim, S. H. (1991). J. Appl. Cryst. 24, 409-411.

Kornfeld, S. & Glaser, L. (1961). J. Biol. Chem. 236, 1791-1794.

Leslie, A. G. W. (1992). CCP4 and ESF-EACMB Newsletter on Protein Crystallography, Number 26

Matthews, B. W. (1968). J. Mol. Biol. 33, 491-497.

Ma, Y., Mills, J. A., Belisle, J. T., Vissa, V., Howell, M., Bowlin, K., Scherman, M. S. & McNeil, M. (1997). Microbiology 143, 937-945

McNeil, M., Daffe, M. & Brennan, P. J. (1990). J. Biol. Chem. 265, 18200-18206.

Melo, A. & Glaser, L. (1965). The Nucleotide Specificity and Feedback Control of Thymidine Diphosphate D-glucose Pyrophosphorylase. J. Biol. Chem. 240, 398-405

Nakano, Y., Suzuki, N., Yoshida, Y., Nezu, T., Yamashita, Y. & Koga, T. (2000). J. Biol. Chem. 275, 6806-6812

Newton, D. T. & Mangroo, D. (1999). Biochem. J. 339, 63-69.

O'Brien, W. E. (1976). Anal Biochem. 76, 423-430.

Otwinowski, Z. & Minor, W. (1996). Processing of X-ray diffraction data collected in oscillation mode. Methods Enzymol. 276, 307-326.

Paule, M. R. & Preiss, J. (1971). J. Biol. Chem. 246, 4602-4609

Rahim, R. et al. (2000). Submitted Sheu, K. F. & Frey, P. A. (1978). J. Biol. Chem. 253, 3378-3380

Sussman, J. L., Lin, D., Jiang, J., Manning, N. O., Prilusky, J., Ritter, O. & Abola, E. E. (1998). Acta Cryst. D54, 1078-1084

Terwilliger; T. C. & Berendzen, J. (1999). Acta Cryst. D55, 849-61

Theorell, H. & Chance, B. (1951). Acta Chem. Scand. 5, 1127

Tong, L. & Rossmann, M. G. (1997). Methods Enzymol. 276, 594-611

Yeh, J. I. & Hol, W. G. (1999). Acta Cryst. D54, 479-480

Yoshida, Y., Nakano, Y., Nezu, T., Yamashita, Y. & Koga, T. (1999). J. Biol. Chem. 274, 16933-16939

1. A method of selecting agents which inhibit the enzyme glucose-1-phosphate thymidylyltransferase (RmlA), said method comprising the steps of:
a) providing a model of the active or regulatory site(s) of RmlA;
b) reviewing the structure of a potential inhibitory agent for at least one of these sites; and
c) analysing the potential interaction of said agent in said site(s).

2. A method as claimed in claim 1 further including the step of selecting an agent which interacts with the active or regulatory site(s) of RmlA.

3. The method as claimed in claim 2 wherein said agent binds to the active or regulatory site(s) of RmlA sufficiently tightly to impede the biosynthesis of rhamnose.

4. The method as claimed in either one of claims 2 and 3 wherein said agent has a negative charge which interacts with Arg 15 and/or Lys 25 of RmlA.

5. The method as claimed in either one of claims 2 and 3 wherein said agent has a thymidyl-like moiety able to interact with Gly 10, Gln 82 and/or Gly 87 of RmlA.

6. The method as claimed in claim 5 wherein said thymidyl-like moiety forms a hydrogen bond with Gly 10, Gln 82 and/or Gly 87 of RmlA.

7. The method as claimed in either one of claims 2 and 3 wherein said agent has a glucose-like moiety able to interact with Asn 111, Gly 146, Glu 161, Val 172 and/or Thr 176 of RmlA.

8. The method as claimed in claim 7 wherein said glucose-like moiety forms a hydrogen bond with Asn 111, Gly 146, Glu 161, Val 172 and/or Thr 176 of RmlA.

9. The method as claimed in any one of claims 1 to 8 wherein said model of RmlA is in the form of a computer data file.

10. The method as claimed in any one of claims 1 to 9 wherein said model is based upon the X-ray crystal co-ordinates of RmlA.

11. The method as claimed in claim 10 wherein said model includes the data for the regulatory site(s) as set out in Annex 1.

12. The method as claimed in claim 10 wherein said model includes the data for the active site(s) as set out in Annex 2.

13. The method as claimed in any one of claims 1 to 12 wherein step b) includes providing a model of the potential inhibitory agent.

14. The method as claimed in claim 13 wherein said model is in the form of a computer data file.

15. The method as claimed in any one of claims 1 to 14 wherein the intermolecular interaction between said agent and the model of the active or regulatory site(s) of RmlA is analysed with the aid of a computer.

16. A purified and crystallised form of the enzyme glucose-1-phosphate thymidylyltransferase (RmlA) obtained from Pseudomonas aeruginosa.

17. Use of the purified and crystallised form of RmlA as claimed in claim 16 to select for inhibitors of said enzyme.

18. Use as claimed in claim 17 wherein said inhibitors inhibit the growth of Pseudomonas aeruginosa.
